(19) United States
(12) Patent Application Publication
(10) Pub. No.: US 2025/0173507 A1
(43) Pub. Date: May 29, 2025

(54) METHOD AND APPARATUS FOR AUTOMATICALLY INPUTTING ELECTRONIC NURSING RECORD USING GENERATIVE ARTIFICIAL INTELLIGENCE

(71) Applicant: Dongkyun LEE, Yongin-si (KR)

(72) Inventor: Dongkyun LEE, Yongin-si (KR)

(21) Appl. No.: 18/900,348

(22) Filed: Sep. 27, 2024

(30) Foreign Application Priority Data
Nov. 24, 2023 (KR) 10-2023-0165004

(51) Int. Cl. G06F 40/284; G06F 40/274; G16H 10/60

(52) U.S. Cl. CPC G06F 40/284; G06F 40/274; G16H 10/60

(57) ABSTRACT

The present disclosure relates to a method for automatically inputting electronic nursing records, comprising: (a) a step of selecting a user interface unit through an electronic nursing record system; (b) a step of inputting text into an input field of the user interface based on the predefined items of nursing notes; (c) a step of cleaning and tokenizing the input text; (d) a step of fine-tuning the text tokenized above; and (e) a step of inputting the text fine-tuned above into the electronic nursing record system, wherein in the fine-tuning step, at least one nursing record data corresponding to the input text can be selected from a nursing record database designated in advance to be used for auto completion or auto correction.

### CROSS-REFERENCE TO RELATED APPLICATIONS

This application claims the benefit of Korean Application No. 10-2023-0165004, filed Nov. 24, 2023, in the Korean Intellectual Property Office. All disclosures of the documents named above are incorporated herein by reference.

### TECHNICAL FIELD

The present disclosure relates to a method of automatically inputting electronic nursing records and an apparatus thereof. More specifically, it relates to a medical record (nursing care) system using generative artificial intelligence that can be utilized in lectures and clinical training sites by prospective medical professional who wish to become medical professionals.

### BACKGROUND

An electronic medical record system refers to the computational processing of medical records of medical support services in a hospital setting. In this regards, when hospital officials enter patient information into the electronic medical record system established within a hospital, they can view various patients' medical records.

An electronic medical record system has been introduced and widely used in most hospitals and clinics. However, it is difficult for healthcare and life science courses and lectures from universities to keep up with changes with respect to hospital information systems of hospitals.

As a result, many medical training programs use handwritten medical records rather than using a computerized system. To solve this problem, an electronic medical record, especially an electronic nursing record, used in hospitals and clinics was made available for educational purposes; however, it still takes a lot of time to input nursing records into the system since it is only a change from handwriting to typing.

Therefore, there is a steady demand for technology that can automatically input nursing notes through artificial intelligence modeling, generative artificial intelligence, or database where the nursing notes of a virtual patient created for educational purposes.

In case of the conventional technology related to an electronic nursing record, it takes a long time to record nursing notes that when medical students actually work at the clinical (hospital) situations, they often have difficulty using a nursing record system, which making them hard to adjust to work. Furthermore, in order to use an electronic nursing record system used in clinical practice for educational purposes, actual patients' personal information have to be used directly. There is still room for further improvement in the educational electronic nursing record system for future nurses.

Therefore, there is a need for a specific method for solving such problems.

### Document of Related Art

### [Patent Document]

### (Patent Document 1) Korean Patent Publication No. 10-2022-0164439 (Published on Dec. 13, 2022)

### SUMMARY

The present disclosure relates to a method and apparatus for automatically inputting electronic nursing records using generative artificial intelligence and it aims to support medical students to practice and use an electronic nursing record and electronic medical record system so that they can adapt to each of different hospital settings faster.

In addition, the purpose of the present disclosure is to improve the accuracy and speed of recording nursing notes by automatically correcting errors and typos according to context in creating an electronic nursing record.

In order to solve the above-mentioned problems, a method of automatically inputting electronic nursing records according to the present disclosure includes (a) a step of selecting a user interface unit through an electronic nursing record system; (b) a step of inputting text into an input field of the user interface based on the predefined items of nursing notes; (c) a step of cleaning and tokenizing the input text; (d) a step of fine-tuning the text tokenized above; and (e) a step of inputting the text fine-tuned above into the electronic nursing record system. In the fine-tuning step, at least one nursing record data corresponding to the input text can be selected from a nursing record database designated in advance to be used for auto completion or auto correction.

As described above, the step of cleaning and tokenizing the input text and the step of fine-tuning the text tokenized above are done using a generative artificial intelligence module.

As described above, the input value of an input field may include at least one of the following contents: medical record; disease code; vital signs; fall risk; blood test results; electrolyte imbalance test results; and prescription history.

As described above, the nursing records include at least one of nursing systems: North American Nursing Diagnosis Association (NANDA); SOAPIE; Focus DAR, International Classification for Nursing Practice (ICNP); narrative notes; and nursing processes, and wherein the method further includes a step of changing and inputting the nursing records through an interface change unit the user interface unit.

As described above, among at least one nursing record data selected from the predefined nursing record database, the nursing record data used to automatically complete or correct the input text is determined according to the predetermined priority or preferences of pre-trained users.

The apparatus that automatically inputs electronic nursing records according to the present disclosure is comprised of (a) a user interface unit that includes an input field based on each predetermined nursing record item; (b) a nursing record database that stores the nursing record data of virtual patients and nursing record data of patients; (c) a control unit that automatically completes or corrects the text inputted to an input field contextually; (d) a communication unit that transmits and receives data about the text from the nursing record database in order to automatically complete and correct them in the control unit wherein the auto completion and auto correction can be accomplished through a generative artificial intelligence module within the control unit.

As described above, it includes an interface change unit capable of changing and inputting the nursing records based on the input preferences contextually wherein it includes at least one of nursing systems: North American Nursing Diagnosis Association (NANDA), SOAPIE, Focus DAR, International Classification for Nursing Practice (ICNP), narrative notes, and nursing processes.

The method and apparatus thereof for automatically inputting electronic nursing records using generative artificial intelligence according to the present disclosure can improve the accuracy and speed of filing nursing notes.

The method and apparatus thereof for automatically inputting electronic nursing records using generative artificial intelligence according to the present disclosure can be used to support nurses with their work and improve adaptability of the nursing students' hands-on clinical experiences.

The method and apparatus thereof for automatically inputting electronic nursing records using generative artificial intelligence according to the present disclosure can be applied to an electronic health record system such as an electronic nursing record (ENR) or electronic medical record (EMR) to improve the system.

The effects of the present disclosure are not limited by the embodiments exemplified above, and more effects may be included in the present disclosure.

### BRIEF DESCRIPTION OF THE DRAWINGS

A more complete understanding the disclosed embodiments of the present disclosure may be better understood by reference to the following description in conjunction with the accompanying drawings, in which the same or like reference numerals designate the same or like elements.

FIG. 1 is a flowchart illustrating a method of automatically inputting electronic nursing records according to an embodiment of the present disclosure.

FIG. 2 is a flowchart illustrating a method of automatically inputting electronic nursing records using generative artificial intelligence according to an embodiment of the present disclosure.

FIG. 3 is a flowchart illustrating the content inputted to the text input fields within the method of automatically inputting electronic nursing records using generative artificial intelligence according to an embodiment of the present disclosure.

FIG. 4 is a flowchart illustrating the content and an input method within the method of automatically inputting electronic nursing records using generative artificial intelligence according to an embodiment of the present disclosure.

FIG. 5 is a diagram illustrating an example of FIG. 4.

FIG. 6 is a conceptual diagram illustrating an apparatus for automatically inputting electronic nursing records using generative artificial intelligence according to an embodiment of the present disclosure.

FIG. 7 is a conceptual diagram illustrating the principle of implementing an apparatus for automatically inputting electronic nursing records using generative artificial intelligence according to an embodiment of the present disclosure.

It is to be understood that that drawings referenced above are not necessarily drawn to scale, but rather present simplified representations of various preferred features illustrating the basic principles of the present disclosure. Certain design features of the present disclosure, for example, including particular dimensions, orientation, location, and shape will be determined in part by the particularly intended applications and environments of use.

Hereinafter, some embodiments of the present disclosure will be described in detail with reference exemplary drawings. In adding reference numerals to components of each drawing, the same elements may have the same reference numerals as much as possible even though they are indicated in different drawings. In addition, in describing the present disclosure, if it is determined that a detailed description of a related known configuration or function may obscure the gist of the present disclosure, the detailed description may be omitted. When ‘includes’, ‘has’, ‘consist’ mentioned in the present disclosure is used, other parts may be added unless ‘only’ is used. In the case where an element is expressed in the singular, it does not exclude a plurality unless indicated otherwise.

In addition, in describing the elements of the present disclosure, terms such as first, second, A, B, (a), (b), and so on may be used. These terms are only for distinguishing the elements from other elements and the essence, order, sequence, or number of corresponding elements is not limited by the terms.

In the description of the potential relationship of the elements, when two or more elements are described as being “connected”, “coupled”, or “accessed”, two or more elements are directly “connected”, “coupled” or “accessed.” It should be understood that, however, two or more elements and other element may be further “interposed” and “connected,” “coupled,” or “accessed.” At this time, other elements may be included in one or more of two or more elements that are “connected”, “coupled” or “accessed” to each other.

In the description of temporal flow relationships related to elements, operation methods, production methods, for example, temporal precedence relationships such as “after,” “followed by,” “following,” “before,” and so on, or a sequential relationship is described, non-continuous cases may be included unless “immediately” or “directly” is used.

Meanwhile, when numerical values or corresponding information (e.g. level) for an element are mentioned, even if there is no explicit description, the numerical value or the corresponding information is based on various factors (such as process factors, internal or external chock, and noise) and it should be interpreted to include a potential error range.

The present disclosure is based on the technology that recommends and produces sentences by using a weighted scoring of the text and frequency of the nursing database sentences using evaluation metrics such as Top-K recommendation or Top-K accuracy when fine-tuning. Hereinafter, the present disclosure will be described in detail with reference to the accompanying drawings.

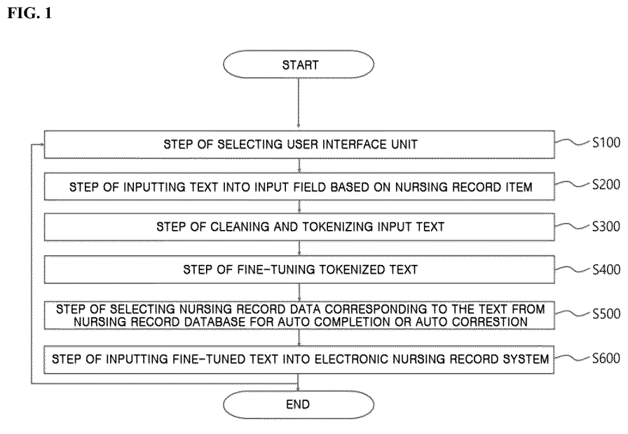

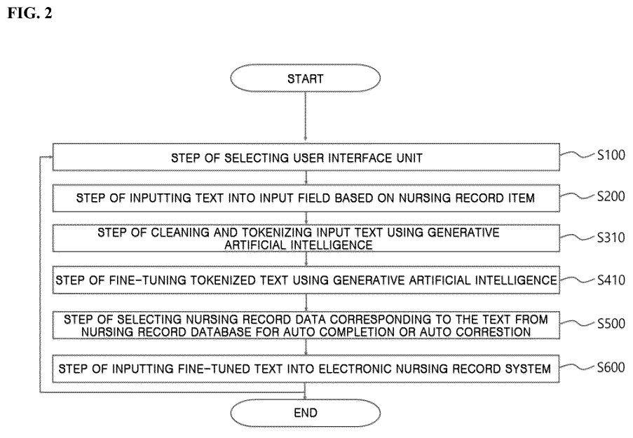

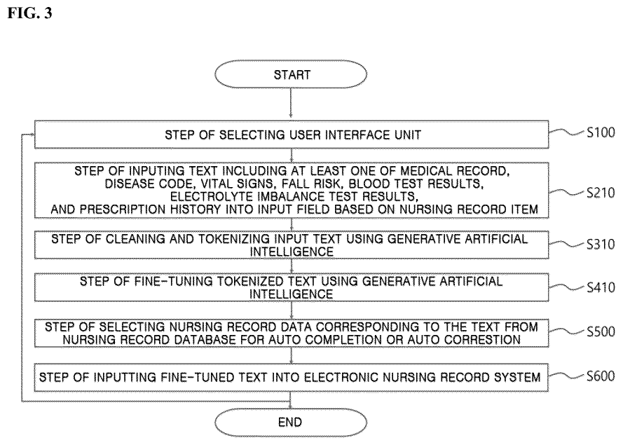

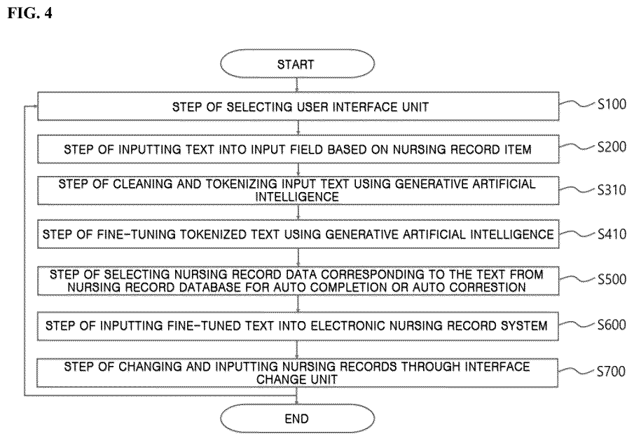

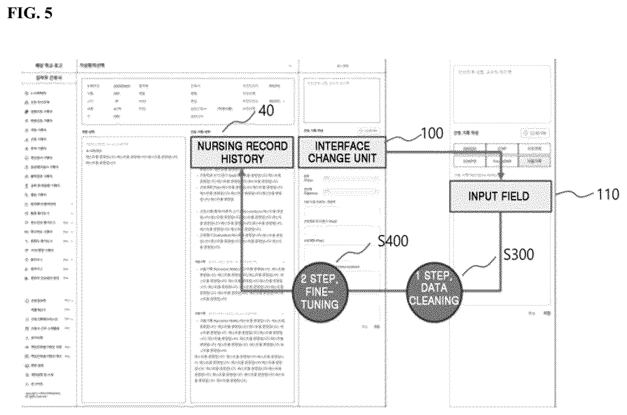

FIG. 1 is a flowchart of a method for automatically inputting electronic nursing records according to an embodiment of the present disclosure; FIG. 2 is a flowchart of a method for automatically inputting electronic nursing records using generative artificial intelligence according to an embodiment of the present disclosure; FIG. 3 is a flowchart illustrating the content inputted to the text entry fields within the method of automatically inputting electronic nursing records using generative artificial intelligence according to an embodiment of the present disclosure; FIG. 4 is a flowchart illustrating the content and an input method within the method of automatically inputting electronic nursing records using generative artificial intelligence according to an embodiment of the present disclosure; and FIG. 5 is a diagram illustrating an example of FIG. 4.

Referring to FIG. 1, a method for automatically inputting electronic nursing records through an apparatus 1 thereof according to an embodiment of the present disclosure includes a step S100 of selecting a user interface 10 through an electronic nursing record system 40. As an example, the user interface unit 10 may have an input scheme based on either North American Nursing Diagnosis Association (NANDA) or International Classification for Nursing Practice (ICNP), but is not limited to this.

In one example, NANDA stands for NANDA International, an international standard for classification of the nursing diagnoses. In other words, NANDA is the abbreviation for North American Nursing Diagnosis Association. NANDA International focuses on defining and standardizing nursing diagnoses in nursing practice. Nursing diagnoses refer to either the nurse's role or the nursing process to evaluate and understand the patient's condition, where they are used to explain the patient's health status and establish a preventive or situational nursing care plan.

Meanwhile, the ICNP stands for International Classification for Nursing Practice, and it corresponds to a classification system of standardized nursing diagnoses and nursing interventions that are used internationally by international organizations for nurses. The ICNP provides a unified language system and structure where experts such as nurses, nurse educators, health information managers, and healthcare experts can classify and understand the nursing care problems.

Next, a step S200 of inputting the text according to an element of the nursing records into a text input field of a user interface unit 10 is disclosed. As an example, the input content entered into the text input field may include at least one of the following contents: medical record; disease code; vital signs; fall risk; blood test results; electrolyte imbalance test results; and prescription history, but is not limited thereto wherein a different value may be inputted or entered depending on the settings.

Next, a step S300 of cleaning and tokenizing the input text and a step S400 of fine-tuning the tokenized text are disclosed.

The steps of tokenizing and/or fine-tuning refer to some kind of cleaning the input text, which are to remove noise from the text and standardize the text in a consistent format.

As an example of cleaning, a process may involve converting the text to lowercase and/or uppercase for English text. Furthermore, it can emphasize the critical information of the text by selecting and eliminating words that appear frequently in the text, but irrelevant. In addition, the input text can be cleaned and refined by removing special characters or using a regular expression, stemming and lemmatization, removing duplicate words, and frequency-based filtering.

Meanwhile, tokenizing is a process of dividing a given text into smaller units (tokens), where these small units generally refer to words, sentences, or paragraphs. In other words, it is to break down given text into the smallest meaningful units. Thus, the input text may be divided into predetermined units such as words, sentences, or paragraphs through a tokenizing process. In this process, in addition to simply dividing the input text into predetermined units of words, sentences, or paragraphs, it removes all or part of recurrent meaningless words in the input text or converts the input text or the structures into a certain format.

Tokenizing is one of preprocessing steps for natural language processing (NLP) and machine learning. Tokenizing serves as an initial step for natural language processing (NLP) where the text data are converted into a form that can be input into a model. The tokenized data are generally used to input a model or in text-based tasks. Technically, this is done using regular expressions, functions for string processing, or special libraries.

The fine-tuning process means that it automatically completes or corrects the text by using a pre-trained neural network model in order to improve the overall clarity and accuracy of the text. In this process, the data that corresponds to the text and the predefined nursing record database are transmitted and received, which are used to automatically complete or correct the text.

Meanwhile, the step S300 of cleaning or tokenizing the input text and the step S400 of fine-tuning the tokenized text can be performed by using a generative artificial intelligence module 21.

A generative artificial intelligence module 21 is a type of artificial intelligence that mainly focuses on analyzing the data and creating a new data file. It learns information or relevant patterns in the given input text, and completes or creates new content based on this. Thus, by tokenizing and/or fine-tuning the input text through generative artificial intelligence, the new text to be inputted to the electronic nursing record system will be derived. Examples of the generative artificial intelligence module may include chatGPT, Bard, HyperCLOVA X, but are not limited to thereto.

Meanwhile, to apply and measure performance of the generative artificial intelligence module, the Top-K accuracy classification model and the Top-K recommendation (preference diagnosis) model may be used. Top-K accuracy is one of the indicators that evaluates performance of the model in multi-class classification problems and measures the proportion of the actual class in the top K model predictions.

In Top-K accuracy, K indicates how many top predictions to consider. For example, top-3 accuracy measures the actual class in the top three model predictions. This is especially useful when the order of the top predictions is irrelevant.

Most deep learning frameworks provide libraries or functions that can calculate Top-K accuracy, and generally allow a user to set a K value to be considered when evaluating the model.

Meanwhile, Top-K recommendation refers to a system that recommends the top K items to the user. Preference diagnosis provides a recommendation model (e.g. Text) based on users' preferences and behaviors, but involves a process of improving or explaining thereof.

In the fine-tuning process, classification models such as Top-K recommendation or Top-K accuracy can be used. For example, when fine-tuning the input text, it can select at least one data (or a nursing record) that corresponds to the input text on the database of predefined nursing records and use this as a standard for auto completion or auto correction. The Top-K recommendation classification model can be used to select one or more data (or a nursing record) to be used as a standard on the database of predefined nursing records.

In addition, for each of the numerous data (or a nursing record) recorded in the database of predefined nursing records, a weight is allocated to each data (or a nursing record) based on how much it is used, that is, the frequency. Here, ‘K’ pieces of data in order of the highest weight or the data (or a nursing record) with the highest weight may be selected as a data file (or a nursing record) that corresponds to the input text. This allows the input text to be automatically completed or corrected using the most frequently used data in the nursing record database.

In addition, among at least one or more of data (or a nursing record) selected from the database of the predefined nursing records, the data (or a nursing record) that is used for auto completion or auto correction of the input text may be determined based on the preferences of pre-trained users or pre-defined prioritization criterion.

Meanwhile, the input text that has been automatically completed or corrected through fine-tuning may be recommended to the user as diagnostic data to be entered into an electronic nursing record system.

Meanwhile, when a user cumulatively receives recommendations of diagnostic data through the system, the user's preferred diagnostic data can be collected in consideration of various factors such as behavior record, evaluations, and history. In this case, fine-tuning of the input text may use the user's preferred diagnostic data collected.

In addition, in order to explain the preferred diagnostic data better, it can add a function to explain the reason why it recommends the particular diagnostic data to a user, which provides transparency of the recommendation system. Furthermore, it can update the preferred diagnostic data when a new feedback is collected by receiving feedbacks in real-time.

Furthermore, it can understand the preferences of each user through personalized preference diagnosis, and personalize the effects of recommendations by adding a function that can provide customized recommendations or explanations to each user.

As an example, the process of building a large language model (LLM) can be accomplished by applying machine learning and deep learning into a system and software using embedded artificial intelligence.

Embedded artificial intelligence (AI) refers to a technology that implements artificial intelligence models in more compact and lightweight devices, and can provide an optimized way to implement artificial intelligence algorithms in mobile devices, sensors, embedded systems, IoT devices, and so on.

Since most embedded systems have limited sources, they utilize memories and computational resources efficiently by making models lightweight, and use the optimization scheme that utilizes hardware accelerators (e.g. GPU, TPU) in order to execute models more efficiently and reduces the size of the models by using the methods such as weight pruning, quantization, and model compression.

Embedded artificial intelligence (AI) is typically executed in an Edge computing environment, which processes and analyzes the data at a location where the data are created (that is, at Edge) enabling real-time responses. Furthermore, it can utilize a hardware acceleration optimized for a specific embedded platform.

These processes allow lightweight models to be effectively applied to an embedded environment, which can execute the functions of artificial intelligence effectively even in real-time or resource-limited environments.

As an example, tokenization can proceed in Natural Language Processing (NLP). NLP is an abbreviation for Natural Language Processing that processes natural languages. By using Natural Language Processing (NLP), text classification is possible, which classifies text documents into multiple categories or classes, and executes tasks such as filtering, sentiment analysis, theme classification, and even machine translation.

In addition, it is possible to run Named Entity Recognition (NER) for text classification and information retrieval within the nursing record database 60 and the electronic nursing record system 40, automatic summarization that concisely summarizes the text or long documents, and suitable text generation that has been confirmed by association analysis unit 220, through the cleaning process.

Next, the present disclosure includes a step S500 of transmitting and receiving the text data between the device that automatically inputs electronic nursing records and the nursing record database in order to automatically complete or correct the text according to context, and a step S600 of delivering nursing notes recorded in the nursing record list in the electronic nursing record system.

As an example, the nursing records include at least one of nursing systems: North American Nursing Diagnosis Association (NANDA), SOAPIE, Focus DAR, International Classification for Nursing Practice (ICNP), narrative notes, and nursing processes.

Thus, the present disclosure may additionally include a step S700 of changing the nursing records according to the user's surrounding environment to be inputted to the user interface 10. As an example, it is possible to change the nursing records to suit the environment of a hospital or university.

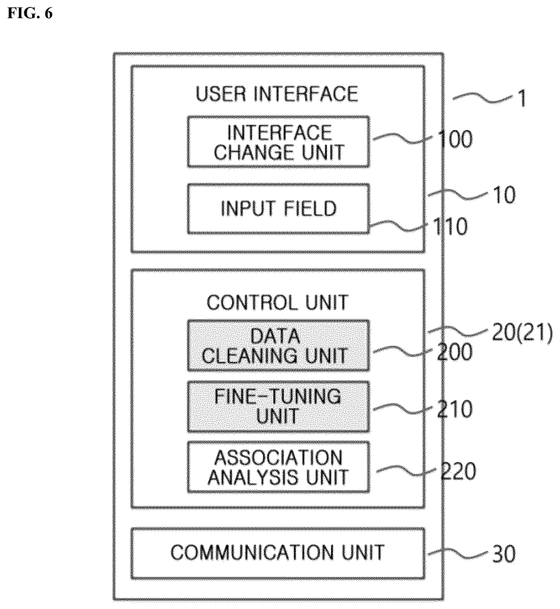

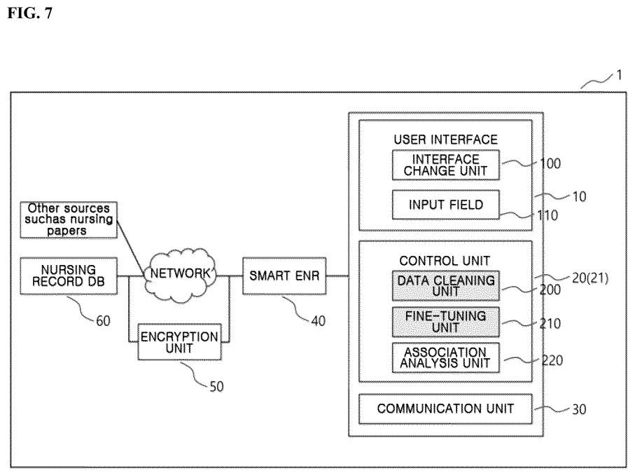

FIG. 6 is a conceptual diagram illustrating an automatic input device for electronic nursing records using generative artificial intelligence in accordance with an embodiment of the present disclosure; FIG. 7 is a conceptual diagram illustrating the principle of implementing an automatic input device for electronic nursing records using generative artificial intelligence in accordance with an embodiment of the present disclosure.

An automatic input device 1 for electronic nursing records in accordance with an embodiment of the present disclosure may include a user interface 10 that has a text input field based on items of predefined nursing records; a nursing record database 60 that stores nursing records of a virtual patient or nursing records of a patient; a control unit 20 that automatically completes or corrects the text in the input field according to context; a nursing record database 60 where auto completion or auto correction can be performed in the control unit 20; a communication unit 30 that can transmit and receive the text data. In addition, auto completion and auto correction features can be managed by a generative artificial intelligence module 21 within the control unit 20.

As an example, the nursing records may include at least one of nursing systems: North American Nursing Diagnosis Association (NANDA), SOAPIE, Focus DAR, International Classification for Nursing Practice (ICNP), narrative notes, and nursing processes; an automatic input device 1 for electronic nursing records may include an interface change unit 100 that enables to change the nursing records according to the user's surrounding environment to be inputted to the user interface 10.

In addition, as can be seen with reference to FIGS. 6 and 7, the method of automatically inputting electronic nursing records using generative artificial intelligence according to an embodiment of the present disclosure can be implemented as an application stored in a computer storage medium or device. A computer may include an automatic input system for electronic nursing records using generative artificial intelligence. An operating system of the computer may be an operating system such as Windows or Macintosh that is installed on general PCs such as desktops or laptops, or a mobile operating system such as iOS or Android that is installed on a mobile terminal such as smartphones or tablet PCs.

A method of automatically inputting electronic nursing records using generative artificial intelligence according to embodiments of the present disclosure may be implemented as an application (that is, computer program) installed by default on a computer or installed by the user, and can be saved (recorded) in a computer-readable storage medium. It may be implemented as hardware components, software components, and/or a combination of hardware components and software components.

As an example, the devices and components described in one or more embodiments can be executed using processors, controllers, arithmetic logic units, digital signal processors, microcomputer, field programmable gate array, programmable logic units, microprocessors, or one or more general computers or special-purpose computers capable of executing and responding to instructions.

For convenience of understanding, a processor may be described as one being used, but a person having ordinary skill in the art, the processing device may include a plurality of processing elements and/or plurality of types of processing.

As an example, a device may include a plurality of processors or a processor and a controller. In addition, other processing configurations, such as parallel processors, are possible.

An application that is saved in a computer storage medium implements an automatic input method for electronic nursing records using generative artificial intelligence may include the following steps: (a) a step of selecting the user interface 10 in the smart electronic nursing record (ENR) program; (b) a step of receiving the text in a text input field 110 tailored to nursing record items in the user interface; (c) a step of cleaning and tokenizing the words or sentences in the text input field 110; (d) a step of fine-tuning the tokenized text using generative artificial intelligence; (e) a step of transmitting the data between the database for auto completion and auto correction; and (f) a step of delivering nursing record written in the nursing record history on the smart electronic nursing record (ENR) program.

In order to execute the automatic input system of electronic nursing records according to embodiments of the present disclosure, which is implemented as a program by reading the program recorded on the storage medium, an application may include codes of computer languages such as C, C++, JAVA, and machine codes that are readable by a computer CPU.

These codes may include function codes with respect to a function that defines functions described above, and the like, and include control codes with respect to an execution procedure necessary for a processor of a computer to execute the functions described above according to a predetermined procedure.

In addition, such codes may further include memory reference-related codes indicating as to which additional information or media required for the computer's processor to execute the above-described functions should be referred from a location (address number) in the computer's internal or external memory.

Meanwhile, when the processor of the computer needs to remotely communicate with any other computers or services in order to execute the functions described above, the code may further include a communication-related code as to how the computer processor remotely communicates with any other computers or services by using a computer's communication module (e.g. wired and/or wireless communication module), and what kind of information or media should be transmitted at the time of communication.

In addition, functional programs and codes and code segments thereof according to embodiments of the present disclosure may be changed or inferred by programmers in the technical field to which the present invention belongs, taking into account the system environment of a computer that reads a storage medium and executes the program.

In addition, the computer-readable storage medium on which the above-described program is recorded may be distributed to a computer system connected through a network so that computer-readable codes can be stored and executed in a distributed manner.

In this case, any one or more of the plurality of distributed computers may execute some of the functions presented above, and transmit the results to one or more of the other distributed computers. Computers that receive the result can also execute some of the functions presented above, and provide the result to other distributed computers as well.

As described above, a computer-readable storage medium having recorded thereon an application for executing an automatic inputting method for electronic nursing records using generative artificial intelligence according to an embodiment of the present disclosure may include ROM, RAM, CD-ROM, magnetic tape, floppy disk, optical disc drive, and the like.

In addition, a computer-readable storage medium having recorded thereon an application for executing an automatic inputting method for electronic nursing records using generative artificial intelligence according to embodiments of the present disclosure may be a storage medium (e.g. hard disk) included in the application provider server which includes an application store server, application, or web server associated with the service, or the application provider server itself, furthermore, it may be another computer that records the program or its storage medium.

In general, a computer-readable storage medium having recorded thereon an application for executing an automatic inputting method for electronic nursing records using generative artificial intelligence may include not only general PCs such as desktops and laptops, but also mobile terminals such as smartphones, tablet PCs, personal digital assistants, and portable terminals. Furthermore, it should be interpreted as all devices capable of computing.

The above description is merely illustrative of the technical idea of the present disclosure, and those of ordinary skill in the technical field to which the present disclosure belongs will be able to make various modifications and variations without departing from the essential characteristics of the present disclosure.

Accordingly, embodiments herein are not intended to limit the technical idea of the present disclosure, but to explain the technical idea, and the scope of the technical idea of the present disclosure is not limited by these embodiments.

The scope of protection of this disclosure should be interpreted by the following claims, and all technical ideas within the scope equivalent thereto should be construed as being included in the scope of the present disclosure.

The method and apparatus for automatically inputting electronic nursing records using generative artificial intelligence can be industrially applicable to various methods and devices for automatically inputting electronic nursing records.

### Description of Reference Signs

1: apparatus for automatically inputting electronic nursing records
10: user interface unit
100: interface change unit
110: text input field
20: control unit
21: generative artificial intelligence module
200: data cleaning unit
210: fine-tuning unit
220: association analysis unit
30: communication unit
40: electronic nursing record system
50: encryption unit
60: database of nursing records

What is claimed is:

1. A method for automatically inputting electronic nursing records, comprising:
a step of selecting a user interface unit through an electronic nursing record system;
a step of inputting text into an input field of a user interface based on the predefined items of nursing notes;
a step of cleaning and tokenizing the input text;
a step of fine-tuning the text tokenized above; and
a step of inputting the text fine-tuned above into the electronic nursing record system;
wherein in the fine-tuning step, at least one nursing record data corresponding to the input text can be selected from a nursing record database designated in advance to be used for auto completion or auto correction.

2. The method for automatically inputting electronic nursing records of claim 1,
wherein the step of cleaning and tokenizing the input text and the step of fine-tuning the text tokenized above are performed using a generative artificial intelligence module.

3. The method for automatically inputting electronic nursing records of claim 2,
wherein the input value of an input field may include at least one of the following contents: medical record; disease code; vital signs; fall risk; blood test results; electrolyte imbalance test results; and prescription history.

4. The method for automatically inputting electronic nursing records of claim 2,
wherein the nursing records include at least one of nursing systems: North American Nursing Diagnosis Association (NANDA); SOAPIE; Focus DAR, International Classification for Nursing Practice (ICNP); narrative notes; and nursing processes; and
wherein the method further includes a step of changing and inputting the nursing records through an interface change unit within the user interface unit.

5. The method for automatically inputting electronic nursing records of claim 1, wherein among at least one or more of data selected from the database of the predefined nursing records, the data that is used for auto completion or auto correction of the input text is determined based on the preferences of pre-trained users or pre-defined prioritization criterion.

6. The automatic input device for electronic nursing records, comprising:
a user interface unit that includes a input field based on each predetermined nursing record item;
a nursing record database that stores the nursing record data of virtual patients and nursing record data of patients;
a control unit that automatically completes or corrects the text inputted to the input field contextually; and
a communication unit that transmits and receives data about the text from the nursing record database in order to automatically complete and correct them in the control unit
wherein the auto completion and auto correction can be accomplished through a generative artificial intelligence module.

7. The automatic input device for electronic nursing records of claim 6,
wherein the automatic input device includes at least one of nursing systems: North American Nursing Diagnosis Association (NANDA), SOAPIE, Focus DAR, International Classification for Nursing Practice (ICNP), narrative notes, and nursing processes; and
wherein the automatic input device further includes an interface change unit capable of changing and inputting the nursing records based on the input preferences contextually.
